(19) United States
(12) Patent Application Publication
(10) Pub. No.: US 2010/0055205 A1
(43) Pub. Date: Mar. 4, 2010

(54) FUNCTIONAL CONSUMABLE COMPOSITIONS FOR PROMOTING SKIN HEALTH AND METHODS FOR USING THE SAME

(76) Inventor: KRISTINA MAINS, Mount Kisco, NY (US)

Correspondence Address: COX SMITH MATTHEWS INCORPORATED, 112 EAST PECAN STREET, SUITE 1800, SAN ANTONIO, TX, 78205-1521, US

(21) Appl. No.: 12/549,589

(22) Filed: Aug. 28, 2009

Related U.S. Application Data
(60) Provisional application No. 61/093,150, filed on Aug. 29, 2008.

(51) Int. Cl. A61K 33/04; A61K 31/352; A61P 17/02

(52) U.S. Cl. 424/702; 514/423

(57) ABSTRACT

A functional consumable composition for promoting skin health may comprise, for each 100% by dry weight, no more than about 0.2% by dry weight catechin and no more than about 2% by dry weight amino acid. The amino acid may comprise at least one of 1-proline and 1-arginine, and the weight % 1-proline to weight % 1-arginine may be greater than about 2. Another functional consumable composition may comprise no more than about 0.2% by dry weight catechin and at least one of lipoic acid, selenium, vitamin E, vitamin B, and vitamin C. A source of the catechin may comprise at least one of white tea solids and epigallocatechin gallate extract. Methods for using such functional consumable compositions are also described.

### CROSS-REFERENCE TO RELATED APPLICATIONS

This application claims priority to U.S. Provisional Patent Application No. 61/093,150 filed on Aug. 29, 2008, the disclosure of which is incorporated herein by reference.

### FIELD

This application relates generally to functional consumable compositions for promoting skin health and methods for using the same.

### BACKGROUND

Studies suggest that no single nutrient is definitive in the promotion of skin health, but rather, several different nutritional compounds appear to play a significant role in the promotion of soft, supple, elastic, and wrinkle-free skin. For example, antioxidants, amino acids, vitamin B, lipoic acid, and selenium may favorably impact skin structure, skin hydration, erythemal index and/or markers of lipid peroxidation.

Numerous scientific studies have highlighted the protective role that antioxidants, including those derived from tea extracts, have on general skin health. Most of the beneficial effects of tea-derived antioxidants, known as catechins, in promoting skin health relate to protection from photo-induced free radicals produced through exposure to ultraviolet radiation. See S K Katiyar et al.; Green Tea and Skin; Archives of Dermatology, 2000; 136 (8): 989-994. However, free radicals are also produced from general oxidative metabolism in the body, and have been implicated in numerous destructive processes within the body. See U.S. Pat. No. 7,015,252 to Fujii et al., which is incorporated in its entirety herein by reference.

While many studies highlight the use of catechins derived from green tea, there is evidence suggesting that white tea extracts have unique properties making them ideal candidates for use in health conscious products. White tea extract is derived from newly formed needles of the tea plant, which are typically dried rapidly after harvesting, as opposed to the slow roast processing used in the processing of green teas. It has been shown that white tea extract is substantially more effective than material derived from green tea extract in suppressing mutagenic activity in the Salmonella assay. See Santana, Rios G et al.; Potent antimutagenic activity of white tea in comparison with green tea; Mutat Res., 2001; 495 (1-2): 61-74.

It appears that polyphenols, which include a number of members of the catechin family, may play a key role in this effect. The catechins are potent antioxidants and, while common to both green and white tea, are included in distinctly different distributions and ratios. While the distribution of catechins may be a key factor in the biology effects of different tea extracts, it is possible that other factors in the tea play at least a supporting role. For example, an “artificial” distribution of catechins, made at the same relative proportions as those in white tea, was found to have significantly lower anti-mutagenic activity than extract from the native tea in the Salmonella mentioned above. Therefore, there appears to be some benefit in using polyphenol catechins derived from white tea.

Vitamin E is a fat soluble compound which has been shown to play several important roles in the body. Vaule, H., et al.; Vitamin E delivery to human skin: studies using deuterated alpha tocopherol measured by APCI LS-MS; Free Rad. Biol. Med., 2004; 36:456-463. For example, vitamin E serves as a lipid soluble antioxidant, in effect helping to terminate free radical initiated lipid peroxidation chain reactions within biological membranes. See Traber Maret G, Attkinson Jeffrey; Vitamin E, Antioxidant and nothing more; Free Radical Biology and Medicine, 2007; 43 (1): 4-15. In terminating a free radical chain reaction, an antioxidant should ideally be available to collide with damaged molecules. A lipid soluble molecule, such as vitamin E, thus provides efficient free radical protection within the cellular membrane, affording complementary protection to other molecules, such as catechins, which may occur with distinct spatial distributions within the cell.

One of the most important effects of reactive free radicals in the skin is the stimulation and over activation of the metalloproteinase class of enzymes. A primary function of these enzymes is the breakdown and rearrangement of collagen. Localized breakdown of collagen is a leading cause for premature skin aging and is of concern in the development of wrinkles. While the pathway leading to metalloproteinase activation has not been completely characterized, oxidation is known to be a strong activator of this class of proteins, thereby lending firm evidence to the causal nature of ultraviolet radiation-induced free radicals in enzyme activation. See Brennan M, et. al.; Matrix metalloproteinase-1 is the major collagenolytic enzyme responsible for collagen damage in UV-irradiated human skin; Photochem Photobiol., 2003; 78 (1): 43-8.

Amino acids also play a protective role in skin health. Collagen is the primary connective element in the body playing a key structural role in connective tissue. This role includes maintenance of structural stability between different layers of the skin. Collagen itself is composed of linear chains of amino acids, the largest percentage of which include glycine and proline. Glycine, the simplest amino acid, is readily available in human diets and easily synthesized in the body. An adequate supply of proline has also been found to be beneficial in the maintenance of healthy skin. See U.S. Pat. No. 6,331,569 to Kisters et al., which is incorporated in its entirety herein by reference. Arginine, like proline, has been implicated as a component enhancing skin health. Arginine is also a consitutent of collagen, and may facilitate healthy skin by serving as a reagent in the natural anabolic biosynthesis of proline, thus serving to subsidize proline ingestion in the diet.

Vitamin B also serves a useful function in promoting skin health. Vitamin B plays a role as a cofactor in many different enzyme-catalyzed reactions, including those associated with the biosynthesis of lipids. See Tanno, O., et al.; Nicotinamide increases biosynthesis of ceramides as well as other stratum corneum lipids to improve the epidermal permeability barrier; Br. J. Dermatol., 2000; 143:524-531. Vitamin C, a potent water soluble antioxidant, serves an important general protective role in the body. However, because vitamin C is readily excreted from the body, it is most effective when administered in a continuous regimen.

Lipoic acid plays several key roles in the body. Most importantly, lipoic acid serves as an important cofactor in aerobic metabolism and is associated with the pyruvate dehydrogenase complex. Additionally, lipoic acid is an effective antioxidant and may play an important role in protecting proteins from reaction with hydroxyl free radicals. See Matsugo S, et al.; Elucidation of antioxidant activity of alpha-lipoic acid toward hydroxyl radical; Biochem Biophys Res Commun., 1995; 208 (1) 161-7.

Selenium plays an important complementary role to the named antioxidants. Selenium serves a key role as a cofactor in several enzymes, including glutathione peroxidase, which catalyze the deactivation of peroxides, a highly reactive source of intracellular oxygen.

While the individual benefits of the foregoing nutrients have been studied, their operational synergy in the area of skin health has not yet been addressed.

### SUMMARY

A functional consumable composition for promoting skin health may comprise, for each 100% by dry weight, no more than about 0.2% by dry weight catechin and no more than about 2% by dry weight amino acid. The amino acid may comprise at least one of 1-proline and 1-arginine, and the weight % 1-proline to weight % 1-arginine may be greater than about 2. Another functional consumable composition may comprise no more than about 0.2% by dry weight catechin and at least one of lipoic acid, selenium, vitamin E, vitamin B, and vitamin C. A source of the catechin may comprise at least one of white tea solids and epigallocatechin gallate extract. Methods for using such functional consumable compositions are also described.

### DETAILED DESCRIPTION

As used herein, the following terms should be understood to have the indicated meanings:

When an item is introduced by “a” or “an,” it should be understood to mean one or more of that item.

The terms “first,” “second,” and the like as used herein do not denote any order, quantity, or importance, but rather are used to distinguish one element from another.

“Catechin” means any one or more of a group of compounds belonging to the flavonoid family.

“Comprises” means includes but is not limited to.

“Comprising” means including but not limited to.

“Having” means including but not limited to.

“Beverage” means any drinkable liquid or semi-liquid, including for example and without limitation, flavored water, soft drinks, fruit drinks, coffee-based drinks, tea-based drinks, juice-based drinks, milk-based drinks, gel drinks, carbonated and non-carbonated drinks, alcoholic and non-alcoholic drinks.

“Food-grade acid” means any acid that is acceptable for use in edible compositions.

“Added water” means water added to a beverage as a component, and does not mean water incidentally added to a beverage through other components. The added water may be specifically purified prior to use using processes well-known in the art such as filtration, deionization, distillation, or reverse osmosis.

“Consumable composition” means any composition that may be orally ingested by a consumer, including without limitation a food, beverage, powder, drink-mix, concentrate, pharmaceutical composition, nutraceutical composition, vitamin, lozenge, dietary supplement, confection, chewing gum, candy, and a combination of any of the foregoing.

“Consumer” means a human or an animal.

“Dry composition” means the composition of a beverage without taking into account any added water.

“Percent by dry weight” means the percent by weight of a component in a dry composition.

“Liquid composition” means the composition of a beverage including any added water.

“Flavors” mean flavoring agents such as natural flavors, artificial flavors, spices, seasonings, and the like. Exemplary flavoring agents may include synthetic flavor oils and flavoring aromatics and/or oils, oleoresins, essences, distillates, and extracts derived from plants, leaves, flowers, fruits, and so forth, and a combination comprising any of the foregoing.

“Flavor potentiator” means a material that can intensify, supplement, modify or enhance the taste and/or aroma perception of a composition without introducing a characteristic taste and/or aroma perception of its own. Flavor potentiators may supplement, modify, or enhance the perception of flavor, sweetness, tartness, umami, kokumi, saltiness, bitterness, and a combination comprising any of the foregoing, for example.

“Nutrient” means any material capable of being metabolized or otherwise used by the body.

The endpoints of all ranges directed to the same component or property are inclusive and independently combinable.

The present application is directed to a beverage composition for promoting skin health. The application is also directed to a method of using a beverage composition for promoting skin health.

In one embodiment, a beverage composition may be formed which contains both catechins and amino acids in the form of 1-proline and 1-arginine. The source of catechins in such embodiment of a beverage composition may include white tea solids, epigallocatechin gallate extract, and a combination comprising any of the foregoing. The chemical composition of one embodiment of such beverage composition may comprise for each 100% by weight of the dry composition, less than about 0.2% by weight catechins and less than about 2% by weight amino acids. Such embodiment of beverage composition may also have a ratio of weight % 1-proline to weight % 1-arginine of at least about 2.

In yet another embodiment, a beverage composition containing both catechins and amino acids in the form of 1-proline and 1-arginine may also contain vitamins or vitamin precursors. Suitable vitamins or vitamin precursors include vitamin A, ascorbic acid (Vitamin C), beta carotene, niacin (Vitamin B₃), riboflavin (Vitamin B₂), thiamin (Vitamin B₁), niacinamide, folate or folic acid, alpha tocopherols or esters thereof, Vitamin D, vitamin E, vitamin K, retinyl acetate, retinyl palmitate, pyridoxine (Vitamin B₆), folic acid (Vitamin B₉, cyanocobalimin (Vitamin B₁₂), pantothenic acid, biotin, and a combination comprising any of the foregoing vitamins.

One embodiment of the method by which an individual may use a beverage composition to promote skin health involves orally administering to the individual an effective amount of a beverage composition containing both catechins and amino acids in the form of 1-proline and 1-arginine. In another embodiment, the beverage composition may also contain vitamins B, C, and E, as well as lipoic acid and selenium.

The consumption of antioxidants in beverage form may yield benefits from issues relating to absorption and clearance. The absorption and clearance of nutrients is a significant concern when considering the delivery of any health product and in particular those that benefit from antioxidant behavior. Many mechanisms of protection from oxidation rely upon the collision of an antioxidant molecule with a free radical constituent in the body. In general, the higher the concentration of antioxidant at the specific cellular location where oxidative damage may occur, the more ameliorative the effect. As such, high concentrations of antioxidants are generally advantageous, balanced by a practical limit to the amount of antioxidants provided to the body. In general, the optimal way to provide a constituent whose benefit depends on concentration is to do so in a near continuous manner. For this reason, a beverage containing antioxidants and other functional ingredients that can be consumed regularly throughout the day may be an effective way to maintain a sufficient concentration of antioxidants and functional ingredients to benefit skin health.

A person having ordinary skill in the art will understand that embodiments of a beverage may contain one or more flavors. Exemplary flavor oils may include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents may include artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, prune, raisin, cola, guarana, neroli, pineapple, apricot, banana, melon, apricot, ume, cherry, raspberry, blackberry, tropical fruit, mango, mangosteen, pomegranate, papaya and so forth. Additional exemplary flavors imparted by a flavoring agent may include a milk flavor, a butter flavor, a cheese flavor, a cream flavor, and a yoghurt flavor; a vanilla flavor; tea or coffee flavors, such as a green tea flavor, an oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamon flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; a nut flavor such as an almond flavor, a hazelnut flavor, a macadamia nut flavor, a peanut flavor, a pecan flavor, a pistachio flavor, and a walnut flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor.

In some embodiments, other flavoring agents may include aldehydes and esters such as cinnamyl acetate, cinnamaldehyde, citral diethylacetal, dihydrocarvyl acetate, eugenyl formate, p-methylamisol, and so forth. Examples of aldehyde flavorings may include acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), and the like.

The flavoring agents may be used in liquid or solid/dried form and may be used individually or in admixture. When employed in dried form, suitable drying means such as spray drying an oil may be used. Alternatively, the flavoring agent may be absorbed onto water-soluble materials, such as cellulose, starch, sugar, maltodextrin, gum arabic and so forth or may be encapsulated. In still other embodiments, the flavoring agent may be adsorbed onto silicas, zeolites, and the like. The techniques for preparing such dried forms are well-known.

In some embodiments, the flavoring agents may be used in many distinct physical forms. Without being limited thereto, such physical forms may include free forms, such as spray dried, powdered, beaded forms, encapsulated forms, emulsions such as caramel or gum arabic emulsions, and a combination comprising at least one of the foregoing physical fowls. The particular amount of the flavoring agent effective for imparting flavor characteristics to the composition may depend upon several factors including the flavor, the flavor impression, and the like.

In another embodiment, a beverage composition may also contain flavor potentiators. Examples of suitable potentiators may include neohesperidin dihydrochalcone, chlorogenic acid, alapyridaine, cynarin, miraculin, glupyridaine, pyridinium-betain compounds, glutamates, such as monosodium glutamate and monopotassium glutamate, neotame, thaumatin, tagatose, trehalose, salts, such as sodium chloride, monoammonium glycyrrhizinate, vanilla extract (in ethyl alcohol), sugar acids, potassium chloride, sodium acid sulfate, hydrolyzed vegetable proteins, hydrolyzed animal proteins, yeast extracts, adenosine monophosphate (AMP), glutathione, nucleotides, such as inosine monophosphate, disodium inosinate, xanthosine monophosphate, guanylate monophosphate, alapyridaine (N-(1-carboxyethyl)-6-(hydroxymethyl)pyridinium-3-ol inner salt), sugar beet extract (alcoholic extract), sugarcane leaf essence (alcoholic extract), curculin, strogin, mabinlin, gymnemic acid, hydroxybenzoic acids, 3-hydroxybenzoic acid, 2,4-dihydroxybenzoic acid, citrus aurantium, vanilla oleoresin, sugarcane leaf essence, maltol, ethyl maltol, vanillin, licorice glycyrrhizinates, compounds that respond to G-protein coupled receptors (T2Rs and T1Rs) and taste potentiator compositions that impart kokumi, as disclosed in U.S. Pat. No. 5,679,397 to Kuroda et al., which is incorporated in its entirety herein by reference, and a combination comprising any of the foregoing potentiators. “Kokumi” refers to materials that impart “mouthfulness” and “good body.”

Sweetener potentiators, which are a type of flavor potentiator, may enhance the taste of sweetness. In some embodiments, exemplary sweetener potentiators may include monoammonium glycyrrhizinate, licorice glycyrrhizinates, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, hydroxybenzoic acids, tagatose, trehalose, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and T1Rs), and a combination comprising any of the foregoing potentiators.

Some embodiments also may include a sweetening agent to provide a sweet taste to the composition. Sweetening agents may include sugar sweeteners, sugarless sweeteners, and a combination comprising any of the foregoing. Sugar sweeteners generally include saccharides. Suitable sugar sweeteners may include mono-saccharides, di-saccharides and poly-saccharides such as sucrose (sugar), dextrose, maltose, dextrin, xylose, ribose, glucose, mannose, galactose, fructose (levulose), lactose, invert sugar, fructo oligo saccharide syrups, partially hydrolyzed starch, corn syrup solids, such as high fructose corn syrup, and a combination comprising any of the foregoing.

Exemplary sugarless sweetening agents may include sugar alcohols (or polyols), such as glycerol, sorbitol, xylitol, mannitol, galactitol, maltitol, hydrogenated isomaltulose (isomalt), lactitol, erythritol, hydrogenated starch hydrolysate, polyglycitol (e.g., syrup or powder), stevia and a combination comprising any of the foregoing. Exemplary hydrogenated starch hydrolysates may include those disclosed in U.S. Pat. Nos. 3,356,811, 4,279,931 and various hydrogenated glucose syrups and/or powders which contain sorbitol, hydrogenated disaccharides, hydrogenated higher polysaccharides, and a combination comprising any of the foregoing. Hydrogenated starch hydrolysates may be prepared by the controlled catalytic hydrogenation of corn syrups. The resulting hydrogenated starch hydrolysates are mixtures of monomeric, dimeric, and polymeric saccharides. The ratios of these different saccharides may give different hydrogenated starch hydrolysates different properties. Mixtures of hydrogenated starch hydrolysates, such as LYCASIN™, a line of commercially available products manufactured by Roquette Freres of France, and HYSTAR™, a line of commercially available products manufactured by Lonza, Inc., of Fairlawn, N.J., also may be useful.

Some embodiments may include high-intensity sweeteners in the composition. Exemplary high-intensity sweeteners may include: (1) water-soluble sweetening agents such as, for example, dihydrochalcones, monellin, steviosides, glycyrrhizin, dihydroflavenol, and sugar alcohols such as sorbitol, mannitol, maltitol, and L-aminodicarboxylic acid aminoalkenoic acid ester amides, such as those disclosed in U.S. Pat. No. 4,619,834, which disclosure is incorporated herein by reference, and a combination comprising any of the foregoing; (2) water-soluble artificial sweeteners such as, for example, soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and a combination comprising any of the foregoing; (3) dipeptide based sweeteners, such as, for example, L-aspartic acid derived sweeteners, such as L-aspartyl-L-phenylalanine methyl ester (Aspartame) and materials described in U.S. Pat. No. 3,492,131, L-alphaaspartyl-N-(2,2,4,4-tetramethyl-3-thietanyl)-D-alaninamide hydrate (Alitame), N—[N-(3,3-dimethylbutyl)-L-aspartyl]-L-phenylalanine 1-methyl ester (Neotame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and a combination comprising any of the foregoing; (4) water-soluble sweeteners derived from naturally occurring water-soluble sweeteners, such as, for example, chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro-4,6,1′,6′-tetradeoxygalacto-sucrose; and 4,6,1′,6′-tetradeoxy-sucrose, and a combination comprising any of the foregoing; (5) protein based sweeteners such as, for example, thaumaoccous danielli (Thaumatin I and II); and (6) the naturally occurring sweetener monatin (2-hydroxy-2-(indol-3-ylmethyl)-4-aminoglutaric acid) and its derivatives.

Many sweetening agents, including some previously discussed, may be categorized as natural sweeteners such as, for example, L-alanine, arabinose, banana extract, carob, cellobiose, corn syrup (including high fructose corn syrup and corn syrup solids), dextrin, dextrose, Dioscoreophyllum cumminsii (Serendipity Berry), erythritol, fructooligosaccharide (FOS), fructose, (including “liquid fructose”), galactose, glucose, glycine, glycyrrhizin, honey, inulin, isomalt, invert sugar, lactitol, lactose, lo han (lo han kuo; lo han guo; lohan guo; lohan kuo), maltitol, maltodextrin, maltose, mannitol, mannose, maple syrup, molasses, partially hydrogenated starch hydrolysate, partially hydrolyzed starch, polydextrose solution, polyglycitol, raftilose, miraculin (Richardella dulcifica (Miracle Berry)), ribose, rice syrup, sorbitol, sorbose, stevia, stevioside, sucralose, sucrose, sugar beets, (dehydrated filaments of), D-tagatose, thaumatin, xylitol, xylose, and a combination comprising any of the foregoing.

Sweetening agents may be used individually or as mixtures and may be used in many distinct physical forms well-known in the art to provide an initial burst of sweetness and/or a prolonged sensation of sweetness. Without being limited thereto, such physical forms may include free forms, such as spray dried, powdered, beaded forms, encapsulated forms, and a combination comprising any of the foregoing. In general, an effective amount of sweetener may be utilized to provide a level of sweetness desired, and this amount may vary with the sweetener selected. Suitable amounts for each type of sweetener may be selected by one of ordinary skill in the art without undue experimentation.

In some embodiments, a beverage composition may include additives such as caffeine, coloring agents (“colorants”, “colorings”), emulsifiers, food-grade acids, minerals, micronutrients, plant extracts, preservatives, salts including buffering salts, stabilizers, thickening agents, medicaments, and a combination comprising any of the foregoing. Those of ordinary skill in the art will understand that certain additives may meet the definition or function according to more than one of the above-listed additive categories.

Exemplary salts may include alkali or alkaline earth metal chlorides, glutamates, and the like. For example, monosodium glutamate, potassium chloride, sodium chloride, and a combination comprising any of the foregoing salts may be used. The salts may be added to the beverage as a flavor potentiator as described above. Food-grade acids for use in certain embodiments of the beverage composition may include, for example, acetic acid, adipic acid, ascorbic acid, butyric acid, citric acid, formic acid, fumaric acid, glyconic acid, lactic acid, malic acid, phosphoric acid, oxalic acid, succinic acid, tartaric acid, and a combination comprising any of the foregoing food-grade acids. The food-grade acid may be added as acidulant to control the pH of the beverage and also to provide some preservative properties; or to stabilize the beverage. The pH of the beverage may also be modified by the addition of food-grade compounds such as ammonium hydroxide, sodium carbonate, potassium carbonate, sodium bicarbonate, and the like, and a combination comprising any of the foregoing. Additionally, the pH of the beverage may be adjusted by the addition of carbon dioxide.

In some embodiments, the tartness of the beverage composition may be varied by selecting and combining acids to provide a desired tartness perception. Some factors to consider in determining a desired tartness include, for example, the acid's dissociation constant, solubility, pH, etc. These variables may be measured by measuring the titratable acidity of the beverage composition.

In one embodiment of a beverage composition, a coloring agent may be used in amounts effective to produce a desired color for the composition. Exemplary coloring agents may include pigments, natural food colors and dyes suitable for food, drug and cosmetic applications. A full recitation of all colorants approved by the United States Food and Drug Administration, together with corresponding chemical structures, may be found in the Kirk-Othmer Encyclopedia of Chemical Technology, 3rd Edition, in volume 5 at pages 857-884, which text is incorporated herein by reference.

As classified by the United States Food, Drug, and Cosmetic Act (21 C.F.R. 73), colors may include those exempt from certification colors (sometimes referred to as natural even though they can be synthetically manufactured) and certified colors (sometimes referred to as artificial), and a combination comprising any of the foregoing. In some embodiments, exemplary colors exempt from certification or natural colors may include, for example, annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161D, caramel (E150 (a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and a combination comprising any of the foregoing.

In some embodiments, exemplary certified colors may include FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and a combination comprising any of the foregoing. In some embodiments, certified colors may include FD&C aluminum lakes, which consist of the aluminum salts of FD&C dyes extended on an insoluble substrate of alumina hydrate. Additionally, in some embodiments, certified colors may be included as calcium salts.

In another embodiment, emulsifiers may be added to the beverage composition to prevent separation of the composition components by keeping ingredients dispersed. Emulsifiers may include molecules which have both a hydrophilic part and a hydrophobic part. Emulsifiers may operate at the interface between hydrophilic and hydrophobic materials of the beverage to prevent separation of the components of the composition. Suitable emulsifiers for use in the described compositions may include, for example, lecithin (e.g., soy lecithin); mono and di-glycerides of long chain fatty acids, specifically saturated fatty acids, and more specifically, stearic and palmitic acid mono- and diglycerides; mono and di-glycerides of acetic acid, citric acid, tartaric acid, or lactic acid; egg yolks; polysorbates (e.g., polysorbate 20, polysorbate 40, polysorbate 60, polysorbate 65, and polysorbate 80), propylene glycol esters (e.g, propylene glycol monostearate); propylene glycol esters of fatty acids; sorbitan esters (e.g., sorbitan monostearates, sorbitan tristearates, sorbitan monolaurate, sorbitan monooleate), Acacia (gum arabic), sucrose monoesters; polyglycerol esters; polyethoxylated glycerols; and the like, and a combination comprising any of the foregoing emulsifiers.

In yet another embodiment, a beverage composition may include certain components (sometimes referred to as hydrocolloids) that act as thickening agents which may impart added “mouth-feel” to the composition. Exemplary thickening agents may include natural and synthetic gums, for example locust bean gum, guar gum, gellan gum, xanthan gum, gum ghatti, modified gum ghatti, tragacanth gum, carrageenan, and the like; natural and modified starches, for example pregelatinized starch (corn, wheat, tapioca), pregelatinized high amylose-content starch, pregelatinized hydrolyzed starches (maltodextrins, corn syrup solids), chemically modified starches such as pregelatinized substituted starches (e.g., octenyl succinate), and the like; cellulose derivatives, for example carboxymethylcellulose, sodium carboxymethylcellulose, and the like; polydextrose; whey or whey protein concentrate; pectin; gelatin; and a combination comprising any of the foregoing thickening agents.

In some embodiments, a beverage composition may include preservatives to provide freshness and to prevent the unwanted growth of bacteria, molds, fungi, or yeast. The addition of a preservative, including antioxidants, may also be used to maintain the composition's color, flavor, or texture. Suitable preservatives may include benzoic acid alkali metal salts (e.g., sodium benzoate), sorbic acid alkali metal salts (e.g., potassium sorbate), ascorbic acid (Vitamin C), citric acid, calcium propionate, sodium erythorbate, sodium nitrite, calcium sorbate, butylated hydroxyanisole (BHA), butylated hydroxytoluene (BHT), ethylenediaminetetraacetic acid (EDTA), tocopherols (Vitamin E), straight chain polyphosphates, and a combination comprising any of the foregoing preservatives.

All patents, patent applications, and other references identified by number herein are incorporated herein by reference in their entirety.

Embodiments of the invention will be further described in connection with the following examples, which are set forth for purposes of illustration only:

### Example 1

A beverage for promoting skin health was prepared by combining catechins in the form of White Tea Solids, additional antioxidants in the form of vitamins C and B, vitamin E, alpha lipoic acid, selenium and the amino acids 1-proline and 1-arginine. Additional components provided, inter alia, coloring and natural flavors. More particularly, a beverage was made having the following dry composition:

Dry Composition of Beverage

| Component | % by Weight |
|---|---|
| Medium Invert Sugar (aqueous solution | 84.58% |
| of sucrose/fructose/glucose) | |
| Honey Granules | 8.544% |
| White Tea Solids | 0.427% |
| l-Proline | 0.901% |
| l-Arginine | 0.094% |
| Alpha Lipoic Acid (99%) | 0.021% |
| Selenium (1%) | 0.034% |
| Vitamin E Acetate (15%) | 0.120% |
| Vitamin B Blend (3, 5, 6, 12) | 0.120% |
| Citric Acid | 1.025% |
| Ascorbic Acid | 0.854% |
| Flavors/colors | 3.277% |

A source of antioxidants in the above example is provided by white tea solids. This ingredient may be derived from the source Camellia sinensis which comprises a distribution of polyphenol catechins as indicated below:

| | % by Weight |
|---|---|
| Component | Camellia Sinensis |
| Catechin gallate | 1.9% |
| Epigallocatechin | 6.8% |
| Catechin | 0.6% |
| Epigallocatechin gallate | 9.4% |
| Gallocatechin | 2.9% |
| Galloacatechin gallate | 0.4% |
| Epicatechin gallate | 3.7% |

Using the above composition, the concentration of individual catechins, expressed as a dry weight percent of the beverage in this example, was as follows:

Dry Composition of Beverage

| Component | % by Weight |
|---|---|
| Catechin gallate | 0.0082% |
| Epigallocatechin | 0.0290% |
| Catechin | 0.0026% |
| Epigallocatechin gallate | 0.0401% |
| Gallocatechin | 0.0124% |
| Galloacatechin gallate | 0.0017% |
| Epicatechin gallate | 0.0158% |

The sum of these individual catechin compositions resulted in a total catechins composition of 0.1098% by weight of dry composition. Amino acids in the form of praline and arginine were 0.995% by weight of the dry composition.

### Example 2

A beverage for promoting skin health was prepared as in Example 1 and had the following dry composition:

Dry Composition of Beverage

| Component | % by Weight |
|---|---|
| Medium Invert Sugar (aqueous solution | 89.04% |
| of sucrose/fructose/glucose) | |
| Honey Granules | 4.200% |
| White Tea Solids | 0.420% |
| l-Proline | 0.886% |
| l-Arginine | 0.092% |
| Alpha Lipoic Acid (99%) | 0.021% |
| Selenium (1%) | 0.034% |
| Vitamin E Acetate (15%) | 0.118% |
| Vitamin B Blend (3, 5, 6, 12) | 0.118% |
| Citric Acid | 1.008% |
| Ascorbic Acid | 0.840% |
| Flavors/colors | 3.221% |

Using the known concentration of individual catechins derived from white tea solids described in Example 1, the concentration of individual catechins, expressed as a dry weight percent of the beverage in this example, was as follows:

Dry Composition of Beverage

| Component | % by Weight |
|---|---|
| Catechin gallate | 0.0079% |
| Epigallocatechin | 0.0286% |
| Catechin | 0.0025% |
| Epigallocatechin gallate | 0.0395% |
| Gallocatechin | 0.0122% |
| Galloacatechin gallate | 0.0017% |
| Epicatechin gallate | 0.0155% |

The sum of these individual catechin compositions resulted in a total catechins composition of 0.1079% by weight of dry composition. Amino acids in the form of proline and arginine were 0.978% by weight of the dry composition.

### Example 3

A beverage for promoting skin health was prepared by combining catechins in the form of White Tea Solids and epigallocatechin gallate, additional antioxidants in the form of vitamins C and B, vitamin E, selenium and the amino acids 1-proline and 1-arginine. Additional components provided, inter alia, coloring and natural flavors. More particularly, a beverage was made having the following dry composition:

Dry Composition of Beverage

| Component | % by Weight |
|---|---|
| Medium Invert Sugar (aqueous solution | 87.42% |
| of sucrose/fructose/glucose) | |
| Honey Granules | 8.830% |
| White Tea Solids | 0.441% |
| l-Proline | 0.933% |
| l-Arginine | 0.097% |
| Selenium (1%) | 0.035% |
| Vitamin E Acetate (15%) | 0.124% |
| Vitamin B Blend (3, 5, 6, 12) | 0.124% |
| Citric Acid | 1.060% |
| Ascorbic Acid | 0.883% |
| Epigallocatechin gallate | 0.021% |
| Flavors/colors | 0.035% |

In this example, two sources of polyphenol catechins were present. In addition to catechins derived from Camellia Sinensis extract, epigallocatechin gallate was also added, such that the concentration of individual catechins, expressed as a dry weight percent of the beverage in this example, was as follows:

Dry Composition of Beverage

| Component | % by Weight |
|---|---|
| Catechin gallate | 0.0084% |
| Epigallocatechin | 0.0300% |
| Catechin | 0.0026% |
| Epigallocatechin gallate | 0.0626% |
| Gallocatechin | 0.0128% |
| Galloacatechin gallate | 0.0018% |
| Epicatechin gallate | 0.0163% |

The sum of these individual catechin compositions resulted in a total catechins composition of 0.1345% by weight of dry composition. Amino acids in the form of proline and arginine were 1.03% by weight of the dry composition.

### Example 4

A beverage for promoting skin health was prepared by combining catechins in the form of White Tea Solids, additional antioxidants in the form of vitamin C, and the amino acids 1-proline and 1-arginine. Additional components provided, inter alia, coloring and natural flavors. More particularly, a beverage was made having the following dry composition:

Dry Composition of Beverage

| Component | % by Weight |
|---|---|
| Medium Invert Sugar (aqueous solution | 82.00% |
| of sucrose/fructose/glucose) | |
| Honey Granules | 9.76% |
| White Tea Solids | 0.49% |
| l-Proline | 1.03% |
| l-Arginine | 0.41% |
| Citric Acid | 1.85% |
| Ascorbic Acid | 0.24% |
| Flavors/colors | 4.21% |

Using the known concentration of individual catechins derived from white tea solids described in Example 1, the concentration of individual catechins, expressed as a dry weight percent of the beverage in this example, was as follows:

Dry Composition of Beverage

| Component | % by Weight |
|---|---|
| Catechin gallate | 0.0093 |
| Epigallocatechin | 0.0333 |
| Catechin | 0.0029 |
| Epigallocatechin gallate | 0.0461 |
| Gallocatechin | 0.0142 |
| Galloacatechin gallate | 0.0020 |
| Epicatechin gallate | 0.0181 |

The sum of these individual catechin compositions resulted in a total catechins composition of 0.1259% by weight of dry composition. Amino acids in the form of proline and arginine were 1.43% by weight of the dry composition.

### Example 5

A beverage for promoting skin health may be prepared as in Example 1 having the following liquid composition:

Liquid Composition of Beverage

| Component | mL | grams | mg/8 oz. | % by Weight |
|---|---|---|---|---|
| Added water, Treated <70 | 956.83 | 954.25 | | 94.1243% |
| Hardness | | | | |
| Medium Invert Sugar, | | 49.50 | | 4.8825% |
| 76.5% solids | | | | |
| Honey Granules | | 5.00 | | 0.4932% |
| White Tea Solids | | 0.25 | 60.0 | 0.0247% |
| l-Proline | | 0.53 | 127.2 | 0.0523% |
| l-Arginine | | 0.06 | 14.4 | 0.0059% |
| Epigallocatechin | | 0.05 | 12.0 | 0.0049% |
| gallate, 94% | | | | |
| Sodium Selenite, | | 0.003 | 0.007 | 0.0003% |
| 1% Selenium | | | | |
| Vitamin E Acetate (15%) | | 0.20 | | 0.0197% |
| Citric Acid | | 0.60 | | 0.0592% |
| Ascorbic Acid | | 0.50 | 120.0 | 0.0493% |
| Flavors/colors | 3.00 | 2.88 | | 0.2837% |

Using the known concentration of individual catechins derived from white tea solids described in Example 1, the concentration of individual catechins, expressed as a dry weight percent of the beverage in this example, is as follows:

Dry Composition of Beverage

| Component | % by Weight |
|---|---|
| Catechin gallate | 0.0079% |
| Epigallocatechin | 0.0286% |
| Catechin | 0.0025% |
| Epigallocatechin gallate | 0.1183% |
| Gallocatechin | 0.0122% |
| Galloacatechin gallate | 0.0017% |
| Epicatechin gallate | 0.0155% |

The sum of these individual catechin compositions results in a total catechins composition of 0.187% by weight of dry composition. Amino acids in the form of proline and arginine are 0.991% by weight of the dry composition.

### Example 6

A beverage for promoting skin health may be prepared as in Example 1 having the following liquid composition:

Liquid Composition of Beverage

| Component | mL | grams | mg/8 oz. | % by Weight |
|---|---|---|---|---|
| Added water, Treated <70 | 959.13 | 956.54 | | 94.38266% |
| Hardness | | | | |
| Medium Invert Sugar, | | 47.70 | | 4.70659% |
| 76.5% solids | | | | |
| Honey Granules | | 5.00 | | 0.49335% |
| White Tea Solids | | 0.25 | 61.7 | 0.02467% |
| l-Proline | | 0.53 | 130.7 | 0.05230% |
| l-Arginine | | 0.06 | 14.8 | 0.00592% |
| Epigallocatechin | | 0.05 | 11.1 | 0.00493% |
| gallate, 90% | | | | |
| Sodium Selenite, | | 0.003 | 0.007 | 0.00030% |
| 1% Selenium | | | | |
| Vitamin E Acetate (15%) | | 0.20 | | 0.01973% |
| Citric Acid | | 0.60 | | 0.05920% |
| Ascorbic Acid | | 0.50 | 123.3 | 0.04934% |
| Flavors/colors | 2.00 | 2.04 | | 0.20102% |

Using the known concentration of individual catechins derived from white tea solids described in Example 1, the concentration of individual catechins, expressed as a dry weight percent of the beverage in this example, is as follows:

Dry Composition of Beverage

| Component | % by Weight |
|---|---|
| Catechin gallate | 0.0084% |
| Epigallocatechin | 0.0299% |
| Catechin | 0.0026% |
| Epigallocatechin gallate | 0.1203% |
| Gallocatechin | 0.0127% |
| Galloacatechin gallate | 0.0018% |
| Epicatechin gallate | 0.0162% |

The sum of these individual catechin compositions results in a total catechins composition of 0.192% by weight of dry composition. Amino acids in the form of proline and arginine are 1.04% by weight of the dry composition.

A beverage composition as described herein may have any desired amount of added water and may be consumed by an individual via oral administration to promote skin health.

### Clinical Study

An independent, single-center, double-blind, randomized, placebo-controlled clinical study was designed and carried out to assess the effectiveness of a twice daily functional beverage on various skin and biochemical parameters in an adult female population. The protocols and results of the clinical study are set forth as presented herein below.

### Study Design

The clinical study was conducted over the course of twelve weeks at the Dallas Research Center of Thomas J. Stephens & Associates, Inc. During the course of the study, subjects were randomly divided into two groups. One group, the “Active Group,” consumed two bottles daily of the beverage described above in EXAMPLE 6, hereinafter referred to as the “Active Treatment,” while the other group, the “Placebo Group,” consumed two bottles daily of the following beverage, hereinafter referred to as the “Placebo Treatment”:

TABLE 1
Liquid Composition of the Placebo Treatment

| Component | mL | grams | % by Weight |
|---|---|---|---|
| Added water, Treated <70 Hardness | 959.91 | 957.32 | 94.5046% |
| Medium Invert Sugar, 76.5% solids | | 47.70 | 4.7088% |
| Honey Granules | | 5.00 | 0.4936% |
| Citric Acid | | 0.93 | 0.0918% |
| Flavors/colors | 2.00 | 2.04 | 0.2011% |

The Placebo Treatment contained a higher concentration of citric acid than the Active Treatment to compensate for the removal of ascorbic acid and to create a similar taste profile between the two Treatments. Subjects were instructed to consume two bottles of their respective Treatment daily (one full bottle in the morning and one full bottle in the afternoon/evening) during the course of the study. Subjects were also counseled by a registered dietitian regarding the importance of keeping their diet constant through the course of the study, and subjects completed a Food Frequency Questionnaire (FFQ) at four time points during the study to monitor diet consistency. Clinic evaluations were conducted on the subjects at the following testing intervals: Baseline (Visit 1), Week 2 (Visit 2), Week 6 (Visit 3), and Week 12 (Visit 4). The evaluations included clinical grading, a series of instrumentation tests, and a self-assessment completed by the subjects.

During the clinical grading portion of the study, a trained evaluator examined the face and volar forearms of the subjects for the following skin condition parameters using a 0-to-9 point grading scale (scale end points are listed in parentheses): fine lines (0=best, 9 worst), coarse wrinkles (0=best, 9 worst), elasticity (0=good recoil properties, 9=poor recoil properties), radiance (0=good luminosity, 9=matte, dull appearance), firmness (0=firm, 9=loose), skin tone (0=rosy skin color, 9=sallowed appearance), skin dryness (0=plump, hydrated appearance, 9=drawn, dry appearance), tactile roughness (0=smooth feel, 9=rough feel), visual roughness (0=smooth, even appearance, 9=rough, uneven appearance), global assessment of mottled pigmentation (0=no hyperpigmentation, 9=pronounced mottled and/or discrete pigmentation), and global assessment of skin health (0=best, 9=worst).

During the instrumentation portion of the study, subjects participated in the following procedures at each of the testing intervals (unless otherwise indicated):

Skicon Measurement. Triplicate measurements were taken on each subject's lower-center, right facial cheek using a skin surface hygrometer to measure the moisture content in the stratum corneum by high frequency conductance (model SKICON-200EX, I.B.S. Co., Ltd., Nagoya, Japan). The measurement is proportional to the dielectric constant of the surface layers of the skin, and increases as the skin becomes more hydrated.

Cutometer Measurement (Excluding Week 2). A single Cutometer measurement was taken on the center of each subject's left facial cheek (model Cutometer SEM 575, Courage and Khazaka Electronic GmbH, Cologne, Germany). The Cutometer determined skin extensibility, resiliency, pure elasticity, and biological elasticity by applying a three hundred (300) mbar negative pressure vacuum perpendicular to the surface of the skin and measuring the resulting skin deformation. Extensibility is inversely proportional to skin firmness, such that a decrease in extensibility indicates improvements in skin firmness. In contrast, resiliency is directly proportional to skin strength and resilient skin has the ability to absorb energy and return (off load the energy) to its original form after being mechanically stretched. An increase in resiliency indicates stronger skin and may suggest an increase in collagen synthesis and/or a reduction in cellular inflammation. Both pure elasticity and biological elasticity refer to the skin's ability to snap back to its original position when stretched, and increases in these elasticity readings suggest that less time is required for the skin to return to its natural state.

Trans-Epidermal Water Loss (TEWL) Measurement. A single TEWL measurement was taken on each subject's left facial cheek to measure the amount of water within the skin that is escaping through the stratum corneum (model Tewameter® TM300, Courage and Khazaka Electronic GmbH, Cologne, Germany). The measurement of this water loss is based on the diffusion principle in an open chamber and the density gradient as measured indirectly by two pairs of sensors located inside a hollow cylinder probe. A decrease in TEWL values, reported in units of g/m²/hr, reflects an improvement in the barrier properties of the skin.

Ultrasound Measurements (Excluding Week 2). An Ultrasound measurement was taken on each subject's left facial side, adjacent to the canthus of the eye, to assess the density and thickness of the facial skin. Measurements were performed using a 50 MHz ultrasonic transducer interfaced to a high frequency ultrasound system (model DUB 6100, Taberna, Pro Medicum AG, Lüneburg Germany). Due to an instrumentation malfunction at the Week 6 time point, ultrasound measurements were not collected for all subjects. Measurements were taken with the probe oriented perpendicular to the body axis while subjects lay supine on a padded patient table. Increases in the density and/or skin thickness measurements suggest a thickening of the epidermal and dermal tissue.

Digital Photography: Raking Light. Raking light photographs were taken of the right side, center, and left side of each subject's face using a custom photostation from Stephens & Associates of Dallas, Tex. Subjects were draped with a black cloth, hair was held away from the face with a headband, and subjects' eyes remained open. Photographs were saved in raw file format (nef) using proprietary software from Nikon Corporation of Tokyo, Japan. After completion of the study, the raking light digital images from one side of the face of each subject were image analyzed for pixel counts, which were then correlated to the relative number, length, width and area of wrinkles.

Biopsy Sample Analysis (Week 12 only). A subgroup of 30 subjects (15 random subjects each from the Active Treatment group and the Placebo Treatment group) underwent four-millimeter (4-mm) trephine punch biopsies taken from the front of the ear on one side of the face. Biopsies were forwarded to ProPath Laboratories, Inc. of Dallas, Tex. and analyzed for glycosaminoglycans (GAG), metalloproteinase 1 and 9 (MMP-1, MMP-9), and collagen. Glycosaminoglycans (GAG) concentration is determined using a colloidal iron stain for mucin and higher concentrations suggest an increase in skin moisturizers. Metalloproteinase 1 and 9 (MMP-1, MMP-9) are capable of degrading extracellular matrix proteins such as collagen and play a role in cell proliferation, migration, differentiation, angiogenesis, apoptosis and host defense. Decreases in the concentration of MMP-1 and MMP-9 suggest increasing levels of collagen. Collagen concentrations are determined by measuring the staining density of total dermal connective tissue fibers using an immunoperoxidase stain. Collagen is responsible for the skin's strength and elasticity, and higher concentrations suggest new collagen synthesis. The scales used to describe the biopsy samples were as follows:

TABLE 2

| | |
|---|---|
| Scale | Description |

glycosaminoglycans (GAG)

| | |
|---|---|
| 0 | None |
| 1 | Mild increase |
| 2 | Moderate increase |
| 3 | Diffuse interstitial mucin |

Metalloproteinase 1 and 9 (MMP-1 and MMP-9)

| | |
|---|---|
| 0 | Normal background staining |
| 1 | Mild staining, <1% of cells |
| 2 | Moderate, <5% of cells |
| 3 | Diffuse >15% cells |

Collagen (% tissue positive for anti-body)

| | |
|---|---|
| 1 | 50 to 65% |
| 2 | 65 to 75% |
| 3 | 75 to 85% |
| 4 | 85 to 95% |
| 5 | 100% |

Antioxidant Protection Measurements. A subgroup of 40 subjects (20 subjects each from the Active Treatment group and the Placebo Treatment group) were selected for additional testing relating to antioxidant protection, using procedures outlined by S. R. Pinnell, J. Murray et al., and C. Oresajo et al. See Pinnell, S. R., Cutaneous photodamage, oxidative stress, and topical antioxidant protection, J. Am. Acad. Dermatol. 2003, 48:1-19; Murray, J. et al., A topical antioxidant solution containing vitamins C and E with ferulic acid protects human skin from sunlight damage and DNA mutations associated with skin cancer, J. Am. Acad. Dermatol. 2008, Vol. 59:418-425; Oresajo, C. et al., Protective effects of a topical antioxidant mixture containing vitamin C, ferulic acid, and phloretin against ultraviolet-induced photodamage in human skin, J. Cosm. Dermatol. 2008, Vol. 7:290-297.

Subjects' backs were examined for scars, birthmarks, moles, vitiligo, keloids, skin abnormalities, tanning, erythema or any other dermal markings that might compromise the test results. At the Baseline visit, each subject received five to seven irradiation exposures on adjacent unprotected skin (US) sites on the lower back in order to determine the subject's natural/inherent MED (minimal erythema dose). At each time point during the study (Baseline, Week 2, Week 6, and Week 12), the subjects received ultraviolet radiation (UVR) in doses of 1.0×, 1.3×, 1.6×, 1.9×, and 2.2× the previously determined MED to an area on the lower back.

UVR was supplied by an artificial source, which has a spectral output in the ultraviolet range comparable to that of the natural solar spectrum (UVB: 290-320 nm and UVA: 320-400 nm). Irradiation was performed with a single port solar simulator equipped with 150-watt xenon arc lamps (model 16S, Solar UV Simulator, Solar Light Co., Philadelphia, Pa.). The desired radiation was obtained by using a combination of the UG-11/1 mm and WG-320 filters that are placed in the radiation path of the solar simulator (Schott Glass Technologies, Mainz, Germany). An adjustable patient stop was used to keep the distance from solar simulator to the radiated surface constant. At a distance of approximately 6.5 cm from the lamp housing, the radiated surface was exposed to a 1.0 cm diameter spot of UVR. The radiation output of the xenon bulb was measured using a UVR intensity meter (model 3D-600, Solar Light Co., Philadelphia, Pa.).

Approximately 16 to 24 hours after each of the irradiation procedures, subjects returned to the clinic and digital photographs were taken of the entire UVR-exposed area under standardized conditions. These photographs were analyzed using a computer-aided colorimetry algorithm to determine a* values, or the degree of redness according to the CIE color standard, for each exposed site. Decreases in a* values are reflected by positive protection factor percentages and suggest that a subject is experiencing increasing levels of antioxidant protection. Increases in a* values are reflected by negative protection factor percentages and suggest that antioxidant protection is decreasing.

During the self-assessment portion of the study, subjects responded to a series of questions relating to perceived changes in the appearance of subjects' skin as well as statements relating to the perceived effectiveness of the subjects' Treatments. For questions relating to perceived changes in the appearance of skin, subjects could answer one of the following: Much Improved, Somewhat Improved, No Change, Somewhat Worse, and Much Worse. For questions relating to the perceived effectiveness of the Treatments, subjects could answer one of the following: Agree Strongly, Agree Somewhat, Neither Agree or Disagree, Disagree Strongly, Disagree Somewhat. The self-assessment questionnaire was administered at Week 2, Week 6, and Week 12.

### Subject Population

Normal, healthy females between the ages of 35 and 65 years were recruited for study participation. Subjects qualified for study participation by having a body mass index (BMI) of 18.5 to 30.0, a Fitzpatrick Skin Classification of Type I through IV, and a Modified Glogau Score of II or III. The scales used for the Fitzpatrick Skin Classification and Modified Glogau Score were as follows:

TABLE 3
Fitzpatrick Skin Classification

| Type | Description |
|---|---|
| I | Always burns easily; never tans |
| II | Always burns easily; tans minimally |
| III | Burns moderately; tans gradually |
| IV | Burns minimally; always tans well |
| V | Rarely burns; tans profusely |
| VI | Never burns; deeply pigmented |

Modified Glogau Score

| Score | Description |
|---|---|
| I | Mild: no keratoses or scarring; little wrinkling |
| II | Moderate: early actinic keratoses - slight yellow skin; |
| | discoloration; early wrinkling - parallel smile line |
| III | Advanced: actinic keratoses - obvious yellow skin; |
| | discoloration with telangiectasia; wrinkling - present at rest |
| IV | Severe: actinic keratoses; skin cancers have occurred; |
| | wrinkling - much cutis laxa of actinic, gravitational, |
| | and dynamic origin |

The table below presents a summary of the demographic information (age, ethnicity, and Fitzpatrick Skin Classification) for all subjects combined and separately for each test material group. For Age, the mean age (in years) and standard deviation have been calculated. For ethnicity and Fitzpatrick type, the number of subjects and percentage of the subject sample in each category are presented:

TABLE 4

| | | | Active | Placebo |
|---|---|---|---|---|
| | | All Subjects | Treatment | Treatment |
| | | (n = 83) | (n = 45) | (n = 38) |
| Age | Mean | 49.00 ± 7.20 | 47.77 ± 7.31 | 50.45 ± 6.89 |
| (Years) | Age ± σ | | | |
| | Minimum | 35.56 | 35.56 | 36.44 |
| | Age | | | |
| | Maximum | 65.49 | 62.40 | 65.49 |
| | Age | | | |
| Ethnicity | Asian | 4 (4.8%) | 3 (6.7%) | 1 (2.1%) |
| | Caucasian | 72 (86.7%) | 37 (82.2%) | 35 (92.1%) |
| | Hispanic | 6 (7.2%) | 4 (8.9%) | 2 (5.3%) |
| | Other | 1 (1.2%) | 1 (2.2%) | |
| Fitzpatrick | Type I | 10 (12.0%) | 6 (13.3%) | 4 (10.5%) |
| Classification | Type II | 48 (57.8%) | 25 (55.6%) | 23 (60.5%) |
| | Type III | 21 (25.3%) | 11 (24.4%) | 10 (26.3%) |
| | Type IV | 4 (4.8%) | 3 (6.7%) | 1 (2.6%) |

One hundred (100) subjects enrolled in the study and seventeen (17) subjects were discontinued due to the following reasons: two (2) subjects voluntarily withdrew, six (6) subjects were unable to attend one or more scheduled visits, one (1) subject was ineligible according to the protocol specifications, one (1) subject was non-compliant, four (4) subjects experienced product distribution errors, and three (3) subjects experienced adverse events. A total of eighty-three (83) female subjects completed the clinical study.

### Statistical Analysis

Mean values for clinical grading parameters and instrumentation measurements at each applicable post-Baseline time point (Week 2, Week 6, Week 12) were calculated and compared to mean Baseline values. Within-Treatment and between-Treatment analyses were conducted as follows:

Clinical Grading and Digital Photography: Raking Light. Within-Treatment analyses were conducted at each time point using a two-sided paired t-test at alpha-0.05. Between-Treatment analyses were conducted at each post-treatment time point utilizing analysis of covariance with the Baseline measurement as the covariate.

Instrumentation measurements (excluding Digital Photography: Raking Light) and Biopsy Sample Collection. Within-Treatment analyses were conducted at each time point using a Student's t-test for paired data. Between-Treatment analyses were conducted at each time point utilizing analysis of covariance with the Baseline measurement as the covariate.

The data from the self-assessment portion of the study were tabulated at each time point. Responses to each question were summarized with the number and percent of subjects with each response shown. For questions with favorable and unfavorable response options, the designated ‘top’ responses were grouped together and statistically compared to the designated ‘lower’ responses. The designated ‘neutral response’ (No Change, Neither Agree nor Disagree) was excluded from response tabulations. Within-Treatment analysis was conducted utilizing a binomial test to determine statistically significant (p≦0.05) differences between the proportion of favorable and unfavorable responses. Between-Treatment comparisons were made using Fisher's Exact Test.

### Results

The data from the clinical grading portion of the study for both the Active and Placebo Treatments were as follows:

TABLE 5

| | | CLINICAL GRADING: ACTIVE GROUP | | | | | | |
|---|---|---|---|---|---|---|---|---|
| | | Baseline | Week 2 | | Week 6 | | Week 12 | |
| | | (n = 48) | (n = 48) | | (n = 45) | | (n = 45) | |
| Face | Fine Lines | 3.29 | 3.30 | (0.3%) | 3.18 [symbol] | (−4.6%) | 2.98 [symbol] | (−10.6%) |
| | Coarse Wrinkles | 3.55 | 3.54 | (−0.2%) | 3.56 | (−0.6%) | 3.48 [symbol] | (−2.7%) |
| | Total Wrinkles | 6.84 | 6.84 | (0.0%) | 6.73 [symbol] | (−2.5%) | 6.46 [symbol] | (−6.5%) |
| | Elasticity | 5.14 | 5.13 | (−0.2%) | 5.04 [symbol] | (−1.9%) | 4.83 [symbol] | (−6.0%) |
| | Radiance | 5.02 | 4.78 [symbol] | (−4.7%) | 4.37 [symbol] | (−14.0%) | 3.73 [symbol] | (−26.4%) |
| | Firmness | 5.66 | 5.63 | (−0.5%) | 5.54 [symbol] | (−2.1%) | 5.27 [symbol] | (−7.0%) |
| | Skin Tone | 4.68 | 4.45 [symbol] | (−4.8%) | 4.11 [symbol] | (−12.1%) | 3.67 [symbol] | (−21.6%) |
| | Skin Dryness | 2.41 | 2.29 [symbol] | (−4.7%) | 1.92 [symbol] | (−18.7%) | 1.23 [symbol] | (−47.8%) |
| | Tactile Roughness | 5.08 | 4.83 [symbol] | (−4.9%) | 4.22 [symbol] | (−17.0%) | 3.04 [symbol] | (−40.1%) |
| | Visual Roughness | 4.71 | 4.58 [symbol] | (−2.6%) | 4.16 [symbol] | (−11.1%) | 3.62 [symbol] | (−22.5%) |
| | Total Roughness | 9.79 | 9.42 [symbol] | (−3.8%) | 8.38 [symbol] | (−14.2%) | 6.67 [symbol] | (−31.7%) |
| | Global Mottled | 3.30 | 3.30 | (0.0%) | 3.22 | (−1.0%) | 3.19 [symbol] | (−2.0%) |
| | Pigmentation | | | | | | | |
| | Global Skin Health | 4.98 | 4.83 [symbol] | (−2.9%) | 4.48 [symbol] | (−10.6%) | 4.06 [symbol] | (−19.0%) |
| Forearm | Fine Lines | 1.36 | 1.35 | (−0.7%) | 1.29 | (−0.8%) | 1.26 | (−3.4%) |
| | Coarse Wrinkles | 0.10 | 0.10 | (0.0%) | 0.11 | (0.0%) | 0.11 | (0.0%) |
| | Total Wrinkles | 1.47 | 1.46 | (−0.7%) | 1.40 | (−0.7%) | 1.37 | (−3.1%) |
| | Elasticity | 4.61 | 4.61 | (0.0%) | 4.60 [symbol] | (−1.1%) | 4.39 [symbol] | (−5.7%) |
| | Radiance | 5.52 | 5.38 [symbol] | (−2.6%) | 5.04 [symbol] | (−9.0%) | 4.56 [symbol] | (−17.8%) |
| | Firmness | 5.15 | 5.15 | (0.0%) | 5.16 [symbol] | (−1.2%) | 4.92 [symbol] | (−5.7%) |
| | Skin Tone | 5.45 | 5.35 [symbol] | (−1.7%) | 5.10 [symbol] | (−6.7%) | 4.70 [symbol] | (−14.0%) |
| | Skin Dryness | 2.77 | 2.70 | (−2.6%) | 2.27 [symbol] | (−18.0%) | 1.72 [symbol] | (−37.7%) |
| | Tactile Roughness | 5.13 | 5.01 [symbol] | (−2.2%) | 4.53 [symbol] | (−12.0%) | 3.91 [symbol] | (−24.1%) |
| | Visual Roughness | 4.86 | 4.84 | (−0.4%) | 4.49 [symbol] | (−7.9%) | 4.10 [symbol] | (−15.9%) |
| | Total Roughness | 9.99 | 9.85 [symbol] | (−1.3%) | 9.02 [symbol] | (−10.0%) | 8.01 [symbol] | (−20.1%) |
| | Global Mottled | 3.88 | 3.88 | (0.0%) | 3.84 | (0.0%) | 3.79 | (−1.4%) |
| | Pigmentation | | | | | | | |
| | Global Skin Health | 4.99 | 4.94 [symbol] | (−1.0%) | 4.63 [symbol] | (−7.9%) | 4.27 [symbol] | (−15.2%) |

[symbol]

Indicates a statistically significant (p ≦ 0.05) decrease (improvement) compared to Baseline

TABLE 6

| | | CLINICAL GRADING: PLACEBO GROUP | | | | | | |
|---|---|---|---|---|---|---|---|---|
| | | Baseline | Week 2 | | Week 6 | | Week 12 | |
| | | (n = 39) | (n = 38) | | (n = 38) | | (n = 37) | |
| Face | Fine Lines | 3.29 | 3.33 | (0.0%) | 3.17 [symbol] | (−2.4%) | 3.09 [symbol] | (−5.7%) |
| | Coarse Wrinkles | 4.00 | 3.97 | (−0.3%) | 4.03 | (−0.6%) | 3.93 [symbol] | (−1.6%) |
| | Total Wrinkles | 7.29 | 7.30 | (−0.1%) | 7.20 [symbol] | (−1.4%) | 7.03 [symbol] | (−3.5%) |
| | Elasticity | 4.94 | 4.92 | (−0.2%) | 4.87 [symbol] | (−1.8%) | 4.59 [symbol] | (−7.8%) |
| | Radiance | 5.04 | 4.92 [symbol] | (−2.3%) | 4.57 [symbol] | (−9.1%) | 4.12 [symbol] | (−17.5%) |
| | Firmness | 5.62 | 5.62 | (0.0%) | 5.54 [symbol] | (−1.1%) | 5.35 [symbol] | (−5.2%) |
| | Skin Tone | 4.55 | 4.47 [symbol] | (−2.0%) | 4.05 [symbol] | (−10.9%) | 3.78 [symbol] | (−16.1%) |
| | Skin Dryness | 2.23 | 2.34 | (2.2%) | 2.18 | (−2.3%) | 1.82 [symbol] | (−20.5%) |
| | Tactile Roughness | 4.88 | 4.68 [symbol] | (−4.0%) | 4.36 [symbol] | (−10.2%) | 3.81 [symbol] | (−22.3%) |
| | Visual Roughness | 4.97 | 4.93 | (−0.7%) | 4.72 [symbol] | (−5.2%) | 4.19 [symbol] | (−16.4%) |
| | Total Roughness | 9.86 | 9.62 [symbol] | (−2.4%) | 9.08 [symbol] | (−7.7%) | 8.00 [symbol] | (−19.3%) |
| | Global Mottled | 3.72 | 3.66 | (0.0%) | 3.67 | (0.0%) | 3.53 | (−1.8%) |
| | Pigmentation | | | | | | | |
| | Global Skin Health | 4.99 | 4.95 | (−0.5%) | 4.72 [symbol] | (−5.2%) | 4.41 [symbol] | (−11.8%) |
| Forearm | Fine Lines | 1.65 | 1.64 | (0.0%) | 1.66 | (0.8%) | 1.69 | (5.0%) |
| | Coarse Wrinkles | 0.46 | 0.47 | (0.0%) | 0.47 | (0.0%) | 0.49 | (0.0%) |
| | Total Wrinkles | 2.12 | 2.12 | (0.0%) | 2.13 | (0.6%) | 2.18 | (3.8%) |
| | Elasticity | 4.83 | 4.80 | (−0.5%) | 4.79 | (−0.8%) | 4.68 [symbol] | (−2.9%) |
| | Radiance | 5.90 | 5.83 [symbol] | (−1.1%) | 5.49 [symbol] | (−6.0%) | 4.97 [symbol] | (−14.8%) |
| | Firmness | 5.15 | 5.14 | (−0.2%) | 5.09 [symbol] | (−1.0%) | 4.93 [symbol] | (−4.1%) |
| | Skin Tone | 5.74 | 5.75 | (−0.2%) | 5.55 [symbol] | (−2.9%) | 5.19 [symbol] | (−9.2%) |
| | Skin Dryness | 3.03 | 3.05 | (−1.6%) | 2.72 [symbol] | (−6.7%) | 2.41 [symbol] | (−19.8%) |
| | Tactile Roughness | 5.38 | 5.20 [symbol] | (−3.6%) | 4.86 [symbol] | (−9.1%) | 4.51 [symbol] | (−16.0%) |
| | Visual Roughness | 5.22 | 5.21 | (−0.2%) | 4.99 [symbol] | (−3.0%) | 4.59 [symbol] | (−10.7%) |
| | Total Roughness | 10.60 | 10.41 [symbol] | (−1.9%) | 9.84 [symbol] | (−6.1%) | 9.11 [symbol] | (−13.4%) |
| | Global Mottled | 4.73 | 4.68 | (0.0%) | 4.70 | (0.0%) | 4.70 | (−0.8%) |
| | Pigmentation | | | | | | | |
| | Global Skin Health | 5.21 | 5.21 | (0.0%) | 4.97 [symbol] | (−3.5%) | 4.68 [symbol] | (−9.4%) |

[symbol]

Indicates a statistically significant (p ≦ 0.05) decrease (improvement) compared to Baseline

The clinical grading results demonstrate a statistically significant (p≦0.05) improvement in skin measurement parameters for the Active Treatment as compared to the Placebo Treatment for the following facial parameters at the indicated time points: Fine Lines at Week 12; Total Wrinkles at Week 12; Radiance at Week 2, Week 6, and Week 12; Skin Tone at Week 2 and Week 12; Skin Dryness at Week 2, Week 6, and Week 12; Tactile Roughness at Week 6 and Week 12; Visual Roughness at Week 2, Week 6, and Week 12; Total Roughness at Week 6 and Week 12; and Global Skin Health at Week 2, Week 6, and Week 12. Additionally, the results demonstrate a statistically significant (p≦0.05) improvement in skin measurement parameters for the Active Treatment as compared to the Placebo Treatment for the following forearm parameters at the indicated time points: Elasticity at Week 12; Radiance at Week 2; Skin Tone at Week 2, Week 6, and Week 12; Skin Dryness at Week 6 and Week 12; Tactile Roughness at Week 12; Visual Roughness at Week 6 and Week 12; Total Roughness at Week 6 and Week 12; and Global Skin Health at Week 2, Week 6, and Week 12. Significant statistical comparisons between the two Treatment groups are summarized in the following table:

TABLE 7

| | | ACTIVE GROUP | | | PLACEBO GROUP | | |
|---|---|---|---|---|---|---|---|
| | | Week | Week | Week | Week | Week | Week |
| | | 2 | 6 | 12 | 2 | 6 | 12 |
| Face | Fine Lines | | [symbol] | [symbol] ♦ | | [symbol] | [symbol] |
| | Coarse Wrinkles | | | [symbol] | | | [symbol] |
| | Total Wrinkles | | [symbol] | [symbol] ♦ | | [symbol] | [symbol] |
| | Elasticity | | [symbol] | [symbol] | | [symbol] | [symbol] |
| | Radiance | [symbol] ♦ | [symbol] ♦ | [symbol] ♦ | [symbol] | [symbol] | [symbol] |
| | Firmness | | [symbol] | [symbol] | | [symbol] | [symbol] |
| | Skin Tone | [symbol] ♦ | [symbol] | [symbol] ♦ | [symbol] | [symbol] | [symbol] |
| | Skin Dryness | [symbol] ♦ | [symbol] ♦ | [symbol] ♦ | | | [symbol] |
| | Tactile Roughness | [symbol] | [symbol] ♦ | [symbol] ♦ | [symbol] | [symbol] | [symbol] |
| | Visual Roughness | [symbol] ♦ | [symbol] ♦ | [symbol] ♦ | | [symbol] | [symbol] |
| | Total Roughness | [symbol] | [symbol] ♦ | [symbol] ♦ | [symbol] | [symbol] | [symbol] |
| | Global Mottled | | | [symbol] | | | |
| | Pigmentation | | | | | | |
| | Global Skin Health | [symbol] ♦ | [symbol] ♦ | [symbol] ♦ | | [symbol] | [symbol] |
| Forearm | Fine Lines | | | | | | |
| | Coarse Wrinkles | | | | | | |
| | Total Wrinkles | | | | | | |
| | Elasticity | | [symbol] | [symbol] ♦ | | | [symbol] |
| | Radiance | [symbol] | [symbol] | [symbol] ♦ | [symbol] | [symbol] | [symbol] |
| | Firmness | | [symbol] | [symbol] | | [symbol] | [symbol] |
| | Skin Tone | [symbol] ♦ | [symbol] ♦ | [symbol] ♦ | | [symbol] | [symbol] |
| | Skin Dryness | | [symbol] ♦ | [symbol] ♦ | | [symbol] | [symbol] |
| | Tactile Roughness | [symbol] | [symbol] | [symbol] ♦ | [symbol] | [symbol] | [symbol] |
| | Visual Roughness | | [symbol] ♦ | [symbol] ♦ | | [symbol] | [symbol] |
| | Total Roughness | [symbol] | [symbol] ♦ | [symbol] ♦ | [symbol] | [symbol] | [symbol] |
| | Global Mottled | | | | | | |
| | Pigmentation | | | | | | |
| | Global Skin Health | [symbol] ♦ | [symbol] ♦ | [symbol] ♦ | | [symbol] | [symbol] |

[symbol]

Indicates a statistically significant (p ≦ 0.05) decrease (improvement) compared to Baseline
♦ Indicates a statistically significant improvement as compared to the other Group

The data from the instrumentation portion of the study for both the Active and Placebo Treatments were as follows:

TABLE 8

| | SKICON MEASUREMENTS | | | | |
|---|---|---|---|---|---|
| | Baseline | Week 2 | Week 6 | | Week 12 |
| Active Group | (n = 48) | (n = 47) | (n = 44) | | (n = 45) |
| | 66.06 | 71.93 (9.1%) | 104.97 [symbol] | (56.7%) | 102.39 [symbol] (53.6%) |
| Placebo Group | (n = 39) | (n = 39) | (n = 38) | | (n = 38) |
| | 80.62 | 74.72 (−7.3%) | 94.71 | (19.1%) | 107.88 [symbol] (35.7%) |

[symbol]

Indicates a statistically significant (p ≦ 0.05) increase (improvement) compared to Baseline

TABLE 9

| | CUTOMETER MEASUREMENTS | | | | | |
|---|---|---|---|---|---|---|
| | | Week | | | | |
| | Baseline | 2 | Week 6 | | Week 12 | |
| Active | | | | | | |
| Group | (n = 47) | | (n = 44) | | (n = 45) | |
| Extensibility | 1.36 | | 1.11 [symbol] | (−17.7%) | 1.27 [symbol] | (−6.4%) |
| Resiliency | 0.70 | | 0.69 | (−1.8%) | 0.71 | (0.3%) |
| Pure | 0.46 | | 0.57 [symbol] | (22.3%) | 0.49 [symbol] | (6.8%) |
| Elasticity | | | | | | |
| Biological | 0.32 | | 0.36 [symbol] | (11.9%) | 0.35 [symbol] | (6.9%) |
| Elasticity | | | | | | |
| Placebo | | | | | | |
| Group | (n = 39) | | (n = 37) | | (n = 38) | |
| Extensibility | 1.34 | | 1.17 [symbol] | (−11.2%) | 1.29 | (−2.8%) |
| Resiliency | 0.72 | | 0.69 [symbol] | (−3.9%) | 0.69 | (−3.9%) |
| Pure | 0.47 | | 0.52 [symbol] | (10.1%) | 0.48 | (1.8%) |
| Elasticity | | | | | | |
| Biological | 0.34 | | 0.34 | (−2.6%) | 0.34 | (−2.9%) |
| Elasticity | | | | | | |

[symbol]

Indicates a statistically significant (p ≦ 0.05) increase (improvement) compared to Baseline [symbol]
Indicates a statistically significant (p ≦ 0.05) decrease (decline) compared to Baseline

TABLE 10

| | TEWL MEASUREMENTS | | | |
|---|---|---|---|---|
| | Baseline | Week 2 | Week 6 | Week 12 |
| Active Group | (n = 48) | (n = 48) | (n = 44) | (n = 45) |
| | 19.86 | 18.67 (−5.9%) | 13.67 [symbol] (−31.5%) | 14.70 [symbol] (−26.3%) |
| Placebo Group | (n = 38) | (n = 39) | (n = 38) | (n = 38) |
| | 19.26 | 19.20 (−0.3%) | 13.15 [symbol] (−32.2%) | 13.81 [symbol] (−28.8%) |

[symbol]

Indicates a statistically significant (p ≦ 0.05) decrease (improvement) compared to Baseline

TABLE 11

| | ULTRASOUND MEASUREMENTS | | | | | |
|---|---|---|---|---|---|---|
| | | Week | | | | |
| | Baseline | 2 | Week 6 | | Week 12 | |
| Active | | | | | | |
| Group | (n = 48) | | (n = 35) | | (n = 45) | |
| Density | 16.96 | | 23.03 [symbol] | (39.9%) | 23.31 [symbol] | (37.3%) |
| Depth | 1438.80 | | 1439.30 | (0.0%) | 1437.70 | (0.0%) |
| [μm] | | | | | | |
| Placebo | | | | | | |
| Group | (n = 39) | | (n = 28) | | (n = 38) | |
| Density | 16.15 | | 17.50 [symbol] | (6.7%) | 18.84 [symbol] | (18.3%) |
| Depth | 1446.50 | | 1456.70 | (0.5%) | 1450.00 | (0.1%) |
| [μm] | | | | | | |

[symbol]

Indicates a statistically significant (p ≦ 0.05) increase (improvement) compared to Baseline

TABLE 12

| | RAKING LIGHT: ACTIVE GROUP | | | | | |
|---|---|---|---|---|---|---|
| | Baseline | Week 2 | | Week 6 | Week 12 | |
| | (n = 47) | (n = 47) | | (n = 45) | (n = 45) | |
| Adjacent: Crow's Feet | | | | | | |
| Number of Wrinkles | 28.17 | 25.73 [symbol] | (−8.6%) | 26.60 (−5.0%) | 25.50 [symbol] | (−9.0%) |
| Total Area | 42.41 | 38.82 [symbol] | (−8.4%) | 39.72 (−6.0%) | 37.99 [symbol] | (−10.1%) |
| Total Length | 133.43 | 121.84 [symbol] | (−8.6%) | 125.97 (−5.1%) | 120.42 [symbol] | (−9.3%) |
| Total Width | 21.45 | 19.63 [symbol] | (−8.4%) | 20.05 (−6.2%) | 19.37 [symbol] | (−9.4%) |
| Beneath: Under-Eye | | | | | | |
| Number of Wrinkles | 8.77 | 8.74 | (−0.2%) | 8.40 (−5.7%) | 7.89 [symbol] | (−11.4%) |
| Total Area | 11.82 | 11.74 | (−0.7%) | 11.36 (−5.7%) | 10.11 [symbol] | (−16.1%) |
| Total Length | 34.97 | 34.64 | (−0.9%) | 33.60 (−5.7%) | 30.84 [symbol] | (−13.4%) |
| Total Width | 7.46 | 7.44 | (−0.2%) | 7.16 (−5.5%) | 6.58 [symbol] | (−13.2%) |
| Total Eye Area | | | | | | |
| Number of Wrinkles | 36.94 | 34.48 [symbol] | (−6.6%) | 35.00 (−5.2%) | 33.39 [symbol] | (−9.5%) |
| Total Area | 54.24 | 50.55 [symbol] | (−6.7%) | 51.08 (−6.0%) | 48.09 [symbol] | (−11.5%) |
| Total Length | 168.39 | 156.48 [symbol] | (−7.0%) | 159.57 (−5.2%) | 151.26 [symbol] | (−10.2%) |
| Total Width | 28.91 | 27.07 [symbol] | (−6.3%) | 27.21 (−6.1%) | 25.95 [symbol] | (−10.4%) |

[symbol]

Indicates a statistically significant (p ≦ 0.05) decrease (improvement) compared to Baseline

TABLE 13

| | RAKING LIGHT: PLACEBO GROUP | | | | | |
|---|---|---|---|---|---|---|
| | Baseline | Week 2 | | Week 6 | Week 12 | |
| | (n = 39) | (n = 39) | | (n = 38) | (n = 38) | |
| Adjacent: Crow's Feet | | | | | | |
| Number of Wrinkles | 28.55 | 27.46 | (−3.8%) | 27.37 (−3.7%) | 27.13 | (−4.6%) |
| Totat Area | 45.88 | 43.43 | (−5.3%) | 43.06 (−5.5%) | 42.18 | (−7.4%) |
| Total Length | 139.21 | 133.84 | (−3.8%) | 131.05 (−5.3%) | 129.51 | (−6.4%) |
| Total Width | 22.43 | 20.96 | (−6.5%) | 21.18 (−5.0%) | 20.81 | (−6.6%) |
| Beneath: Under-Eye | | | | | | |
| Number of Wrinkles | 9.58 | 8.82 | (−7.9%) | 9.21 (−4.4%) | 8.59 [symbol] | (−10.8%) |
| Total Area | 13.12 | 11.79 | (−10.1%) | 12.46 (−5.4%) | 11.22 [symbol] | (−14.8%) |
| Total Length | 38.09 | 35.16 | (−7.6%) | 36.24 (−5.4%) | 33.34 [symbol] | (−12.9%) |
| Total Width | 8.54 | 7.46 [symbol] | (−12.5%) | 8.12 (−5.0%) | 7.43 [symbol] | (−13.0%) |
| Total Eye Area | | | | | | |
| Number of Wrinkles | 38.13 | 36.28 | (−4.8%) | 36.58 (−3.9%) | 35.72 | (−6.2%) |
| Total Area | 59.00 | 55.22 | (−6.4%) | 55.51 (−5.5%) | 53.40 [symbol] | (−9.1%) |
| Total Length | 177.29 | 169.00 | (−4.6%) | 167.29 (−5.3%) | 162.85 [symbol] | (−7.8%) |
| Total Width | 30.96 | 28.42 [symbol] | (−8.2%) | 29.30 (−5.0%) | 28.25 [symbol] | (−8.4%) |

[symbol]

Indicates a statistically significant (p ≦ 0.05) decrease (improvement) compared to Baseline

TABLE 14

| | | BIOPSY SAMPLE ANALYSIS | | | |
|---|---|---|---|---|---|
| | | Baseline | Week 12 | | |
| Active Group | GAG | 1.00 | 1.00 | | (0.0%) |
| (n = 14) | MMP1 | 0.36 | 0.29 | | (−20.0%) |
| | MMP9 | 1.71 | 1.43 | | (−16.6%) |
| | MMP (MMP1 + | 2.07 | 1.71 | | (−17.2%) |
| | MMP9) | | | | |
| | Collagen 1 | 2.00 | 2.00 | | (0.0%) |
| Placebo Group | GAG | 1.00 | 1.00 | | (0.0%) |
| (n = 13) | MMP1 | 0.31 | 0.31 | | (0.0%) |
| | MMP9 | 1.62 | 1.31 | [symbol] | (−19.0%) |
| | MMP (MMP1 + | 1.92 | 1.62 | [symbol] | (−16.0%) |
| | MMP9) | | | | |
| | Collagen 1 | 2.00 | 2.00 | | (0.0%) |

[symbol]

Indicates a statistically significant (p ≦ 0.05) decrease (improvement) compared to Baseline

TABLE 15

| | | ANTIOXIDANT PROTECTION | | |
|---|---|---|---|---|
| | | MEASUREMENTS | | |
| | | Increase from | | |
| | | Unexposed | Baseline | Protection |
| | | (Adjusted for MED) | (Control) | Factor (%) |
| Active Group | Week 2 | 7.67 | 10.04 | 23.6 |
| | Week 6 | 10.80 | 11.23 | 3.86 |
| | Week 12 | 7.76 | 10.07 | 22.87 |
| Placebo Group | Week 2 | 7.40 | 9.28 | 20.19 |
| | Week 6 | 9.51 | 8.82 | −7.87 |
| | Week 12 | 7.39 | 9.21 | 19.72 |

The instrumentation results demonstrate a statistically significant (p≦0.05) improvement for the Active Group as compared to the Placebo Group for the following tests at the indicated time points: Cutometer, Biological Elasticity at Week 6; and Ultrasound, Density at Week 6 and Week 12. Significant statistical comparisons between the two Treatment groups are summarized in the following table:

TABLE 16

| | | Active Group | | | Placebo Group | | |
|---|---|---|---|---|---|---|---|
| | | Week | Week | Week | Week | Week | Week |
| | | 2 | 6 | 12 | 2 | 6 | 12 |
| Cutometer | Extensibility | | [symbol] | [symbol] | | [symbol] | |
| | Resiliency | | | | | [symbol] | |
| | Pure Elasticity | | [symbol] | [symbol] | | [symbol] | |
| | Biological | | [symbol] ♦ | [symbol] | | | |
| | Elasticity | | | | | | |
| Ultrasound | Density | | [symbol] ♦ | [symbol] ♦ | | [symbol] | [symbol] |
| | Depth | | | | | | |

[symbol]

Indicates a statistically significant (p ≦ 0.05) increase (improvement) compared to Baseline [symbol]
Indicates a statistically significant (p ≦ 0.05) decrease (decline) compared to Baseline
♦ Indicates a statistically significant improvement as compared to the other Group

The data from the self-assessment portion of the study for both the Active and Placebo Treatments were as follows:

TABLE 17

| | | CHANGES IN SKIN APPEARANCE: | |
|---|---|---|---|
| | | ACTIVE GROUP | |
| | | Much Improved, | Much Worse, |
| | | Somewhat Improved | Somewhat Worse |
| Overall Skin | Week 2 | * 17 (35.4%) | 3 (6.2%) |
| Health | Week 6 | * 30 (66.6%) | 1 (2.2%) |
| | Week 12 | * 31 (70.4%) | 1 (2.2%) |
| Skin | Week 2 | * 18 (37.5%) | 5 (10.4%) |
| Dryness | Week 6 | * 27 (60.0%) | 1 (2.2%) |
| | Week 12 | * 28 (63.6%) | 0 (0.0%) |
| Skin | Week 2 | * 20 (41.6%) | 2 (4.1%) |
| Hydration | Week 6 | * 28 (62.2%) | 0 (0.0%) |
| | Week 12 | * 31 (70.4%) | 0 (0.0%) |
| Skin | Week 2 | * 21 (43.7%) | 1 (2.0%) |
| Smoothness | Week 6 | * 28 (62.2%) | 0 (0.0%) |
| | Week 12 | * 30 (68.1%) | 0 (0.0%) |
| Skin | Week 2 | * 21 (43.7%) | 1 (2.0%) |
| Softness | Week 6 | * 26 (57.7%) | 0 (0.0%) |
| | Week 12 | * 30 (68.1%) | 0 (0.0%) |
| Skin | Week 2 | * 12 (25.0%) | 0 (0.0%) |
| Elasticity | Week 6 | * 23 (51.1%) | 0 (0.0%) |
| | Week 12 | * 24 (54.5%) | 0 (0.0%) |
| Skin | Week 2 | * 15 (31.2%) | 0 (0.0%) |
| Firmness | Week 6 | * 20 (44.4%) | 0 (0.0%) |
| | Week 12 | * 27 (61.3%) | 0 (0.0%) |

* Indicates a statistically significant (p ≦ 0.05) proportion of subjects responded positively

TABLE 18

| | | LEVEL OF AGREEMENT: | | |
|---|---|---|---|---|
| | | ACTIVE GROUP | | |
| | | Agree Strongly, | | Disagree Strongly, |
| | | Agree Somewhat | | Disagree Somewhat |
| My skin looks | Week 2 | * 18 | (37.5%) | 2 (4.1%) |
| visibly | Week 6 | * 26 | (57.7%) | 1 (2.2%) |
| healthier | Week 12 | * 30 | (68.1%) | 1 (2.2%) |
| My skin feels | Week 2 | * 22 | (45.8%) | 1 (2.0%) |
| healthier | Week 6 | * 31 | (68.8%) | 0 (0.0%) |
| | Week 12 | * 28 | (63.6%) | 0 (0.0%) |
| This product | Week 2 | * 12 | (25.0%) | 0 (0.0%) |
| revitalizes | Week 6 | * 16 | (35.5%) | 0 (0.0%) |
| stressed/tired skin | Week 12 | * 22 | (50.0%) | 1 (2.2%) |
| This product gives | Week 2 | * 13 | (27.0%) | 0 (0.0%) |
| my skin the | Week 6 | * 19 | (42.2%) | 0 (0.0%) |
| natural glow of | Week 12 | * 22 | (50.0%) | 0 (0.0%) |
| healthy skin | | | | |
| My skin looks more | Week 2 | * 10 | (20.8%) | 1 (2.0%) |
| radiant | Week 6 | * 20 | (44.4%) | 0 (0.0%) |
| | Week 12 | * 23 | (52.2%) | 0 (0.0%) |
| My skin looks | Week 2 | 9 | (18.7%) | 2 (4.1%) |
| younger | Week 6 | * 13 | (28.8%) | 0 (0.0%) |
| | Week 12 | * 17 | (38.6%) | 1 (2.2%) |
| My skin looks more | Week 2 | 9 | (18.7%) | 3 (6.2%) |
| luminous | Week 6 | * 16 | (35.5%) | 0 (0.0%) |
| | Week 12 | * 20 | (45.4%) | 1 (2.2%) |
| I look more | Week 2 | 9 | (18.7%) | 2 (4.1%) |
| beautiful | Week 6 | * 9 | (20.0%) | 1 (2.2%) |
| | Week 12 | * 18 | (40.9%) | 2 (4.5%) |
| I feel more | Week 2 | 9 | (18.7%) | 3 (6.2%) |
| beautiful | Week 6 | * 13 | (28.8%) | 1 (2.2%) |
| | Week 12 | * 21 | (47.7%) | 2 (4.5%) |
| My skin feels more | Week 2 | * 21 | (43.7%) | 3 (6.2%) |
| hydrated | Week 6 | * 29 | (64.4%) | 0 (0.0%) |
| | Week 12 | * 31 | (70.4%) | 0 (0.0%) |
| My skin feels | Week 2 | * 21 | (43.7%) | 1 (2.0%) |
| smoother | Week 6 | * 30 | (66.6%) | 0 (0.0%) |
| | Week 12 | * 30 | (68.1%) | 0 (0.0%) |
| My skin feels | Week 2 | * 21 | (43.7%) | 1 (2.0%) |
| softer | Week 6 | * 29 | (64.4%) | 0 (0.0%) |
| | Week 12 | * 30 | (68.1%) | 0 (0.0%) |
| My skin feels | Week 2 | * 15 | (31.2%) | 1 (2.0%) |
| firmer | Week 6 | * 19 | (42.2%) | 0 (0.0%) |
| | Week 12 | * 27 | (61.3%) | 0 (0.0%) |
| My skin feels | Week 2 | 11 | (22.9%) | 3 (6.2%) |
| tighter | Week 6 | * 14 | (31.1%) | 1 (2.2%) |
| | Week 12 | * 22 | (50.0%) | 0 (0.0%) |

* Indicates a statistically significant (p ≦ 0,05) proportion of subjects responded positively

TABLE 19

| | | CHANGES IN SKIN APPEARANCE: | |
|---|---|---|---|
| | | PLACEBO GROUP | |
| | | Much Improved, | Much Worse, |
| | | Somewhat Improved | Somewhat Worse |
| Overall Skin | Week 2 | * 12 (30.7%) | 2 (5.1%) |
| Health | Week 6 | * 32 (84.2%) | 0 (0.0%) |
| | Week 12 | * 35 (92.1%) | 0 (0.0%) |
| Skin | Week 2 | * 14 (35.8%) | 2 (5.1%) |
| Dryness | Week 6 | * 28 (73.6%) | 2 (5.2%) |
| | Week 12 | * 30 (78.9%) | 2 (5.2%) |
| Skin | Week 2 | * 16 (42.1%) | 0 (0.0%) |
| Hydration | Week 6 | * 31 (81.5%) | 1 (2.6%) |
| | Week 12 | * 35 (92.1%) | 0 (0.0%) |
| Skin | Week 2 | * 21 (53.8%) | 0 (0.0%) |
| Smoothness | Week 6 | * 34 (89.4%) | 0 (0.0%) |
| | Week 12 | * 35 (92.1%) | 0 (0.0%) |
| Skin | Week 2 | * 20 (51.2%) | 0 (0.0%) |
| Softness | Week 6 | * 33 (86.8%) | 0 (0.0%) |
| | Week 12 | * 32 (84.2%) | 0 (0.0%) |
| Skin | Week 2 | * 9 (23.0%) | 0 (0.0%) |
| Elasticity | Week 6 | * 21 (55.2%) | 0 (0.0%) |
| | Week 12 | * 30 (78.9%) | 0 (0.0%) |
| Skin | Week 2 | * 10 (25.6%) | 0 (0.0%) |
| Firmness | Week 6 | * 19 (50.0%) | 0 (0.0%) |
| | Week 12 | * 28 (73.6%) | 0 (0.0%) |

* Indicates a statistically significant (p ≦ 0.05) proportion of subjects responded positively

TABLE 20

| | | LEVEL OF AGREEMENT: | |
|---|---|---|---|
| | | PLACEBO GROUP | |
| | | Agree Strongly, | Disagree Strongly, |
| | | Agree Somewhat | Disagree Somewhat |
| My skin looks | Week 2 | * 15 (38.4%) | 3 (7.6%) |
| visibly | Week 6 | * 31 (81.5%) | 0 (0.0%) |
| healthier | Week 12 | * 32 (84.2%) | 0 (0.0%) |
| My skin feels | Week 2 | * 19 (50.0%) | 0 (0.0%) |
| healthier | Week 6 | * 33 (86.8%) | 0 (0.0%) |
| | Week 12 | * 36 (94.7%) | 0 (0.0%) |
| This product | Week 2 | * 11 (28.2%) | 0 (0.0%) |
| revitalizes | Week 6 | * 22 (57.8%) | 0 (0.0%) |
| stressed/tired skin | Week 12 | * 23 (60.5%) | 0 (0.0%) |
| This product gives | Week 2 | * 10 (25.6%) | 0 (0.0%) |
| my skin the | Week 6 | * 23 (60.5%) | 0 (0.0%) |
| natural glow of | Week 12 | * 25 (65.7%) | 0 (0.0%) |
| healthy skin | | | |
| My skin looks more | Week 2 | * 14 (35.8%) | 0 (0.0%) |
| radiant | Week 6 | * 22 (57.8%) | 1 (2.6%) |
| | Week 12 | * 22 (57.8%) | 0 (0.0%) |
| My skin looks | Week 2 | * 10 (25.6%) | 2 (5.1%) |
| younger | Week 6 | * 17 (44.7%) | 1 (2.6%) |
| | Week 12 | * 22 (57.8%) | 0 (0.0%) |
| My skin looks more | Week 2 | * 13 (33.3%) | 2 (5.1%) |
| luminous | Week 6 | * 16 (42.1%) | 1 (2.6%) |
| | Week 12 | * 20 (52.6%) | 0 (0.0%) |
| I look more | Week 2 | 7 (17.9%) | 1 (2.5%) |
| beautiful | Week 6 | * 13 (34.2%) | 1 (2.6%) |
| | Week 12 | * 15 (39.4%) | 0 (0.0%) |
| I feel more | Week 2 | * 16 (41.0%) | 2 (5.1%) |
| beautiful | Week 6 | * 20 (52.6%) | 1 (2.6%) |
| | Week 12 | * 18 (48.6%) | 0 (0.0%) |
| My skin feels more | Week 2 | * 20 (51.2%) | 1 (2.5%) |
| hydrated | Week 6 | * 32 (84.2%) | 1 (2.6%) |
| | Week 12 | * 36 (94.7%) | 0 (0.0%) |
| My skin feels | Week 2 | * 21 (53.8%) | 0 (0.0%) |
| smoother | Week 6 | * 34 (89.4%) | 0 (0.0%) |
| | Week 12 | * 35 (92.1%) | 0 (0.0%) |
| My skin feels | Week 2 | * 20 (51.2%) | 0 (0.0%) |
| softer | Week 6 | * 33 (86.8%) | 0 (0.0%) |
| | Week 12 | * 33 (86.8%) | 0 (0.0%) |
| My skin feels | Week 2 | * 10 (25.6%) | 1 (2.5%) |
| firmer | Week 6 | * 18 (47.3%) | 0 (0.0%) |
| | Week 12 | * 28 (73.6%) | 0 (0.0%) |
| My skin feels | Week 2 | * 8 (20.5%) | 1 (2.5%) |
| tighter | Week 6 | * 16 (42.1%) | 3 (7.8%) |
| | Week 12 | * 24 (63.1%) | 2 (5.2%) |

* Indicates a statistically significant (p ≦ 0.05) proportion of subjects responded positively

The self-assessment results suggest that there were no statistically significant (p 0.05) differences in the proportion of positive to negative responses between the Active and Placebo Groups.

### CONCLUSIONS

During the clinical study, the Active Group consumed a functional composition comprising no more than about 0.2% by dry weight catechin and no more than about 2% by dry weight amino acid, wherein the amino acid comprised at least one of 1-proline and 1-arginine, and wherein a source of the catechin comprised at least one of white tea solids and epigallocatechin gallate extract. The Active Treatment was administered orally as a functional beverage.

The Active Group experienced a statistically significant (p≦0.05) improvement in skin measurement parameters as compared to the Placebo Group for the following clinical grading parameters: (1) Facial parameters, including Fine Lines, Total Wrinkles, Radiance, Skin Tone, Skin Dryness, Tactile Roughness, Visual Roughness, Total Roughness, and Global Skin Health, and (2) Forearm parameters, including Elasticity, Radiance, Skin Tone, Skin Dryness, Tactile Roughness, Visual Roughness, Total Roughness, and Global Skin Health. Additionally, the Active Group experienced a statistically significant (p≦0.05) improvement in skin measurement parameters as compared to the Placebo Group for the following instrumentation measurements: (1) Cutometer measurement, including Biological Elasticity, and (2) Ultrasound measurement, including Density.

Although the Active Group consumed a functional composition comprising amino acids, it is envisioned that functional consumable compositions may be made and consumed which do not comprise amino acids, but rather comprise no more than about 0.2% by dry weight catechin and at least one of lipoic acid, selenium, vitamin E, vitamin B, and vitamin C, wherein a source of the catechin comprises at least one of white tea solids and epigallocatechin gallate extract. It is envisioned that such functional consumable compositions may also be beneficial to skin health. A method for promoting skin health in a consumer may comprise orally administering to the consumer an effective amount of any consumable composition as described herein.

Although the examples given herein pertain to beverage compositions, it is envisioned that functional consumable compositions may be made and consumed in any desired form. Additionally, although the clinical study reported herein involved human subjects, consumable compositions as described herein may also be consumed by animal subjects.

Although the foregoing specific details describe certain embodiments of this invention, persons reasonably skilled in the art will recognize that various changes may be made in the details of this invention without departing from the spirit and scope of the invention as defined in the appended claims and considering the doctrine of equivalents. Therefore, it should be understood that this invention is not to be limited to the specific details shown and described herein.

What is claimed is:

1. A functional consumable composition comprising:
no more than about 0.2% by dry weight catechin; and
no more than about 2% by dry weight amino acid;
wherein said amino acid comprises at least one of 1-proline and 1-arginine;
wherein a source of said catechin comprises at least one of white tea solids and epigallocatechin gallate extract.

2. The functional consumable composition of claim 1 wherein said amino acid comprises 1-proline and 1-arginine.

3. The functional consumable composition of claim 2 wherein the ratio of weight % 1-proline to weight % 1-arginine is greater than about 2.

4. The functional consumable composition of claim 1 wherein said source of said catechin comprises white tea solids and epigallocatechin gallate extract.

5. The functional consumable composition of claim 1 further comprising at least one of lipoic acid, selenium, vitamin E, vitamin B, and vitamin C.

6. The functional consumable composition of claim 1 further comprising lipoic acid, selenium, vitamin E, vitamin B and vitamin C.

7. A functional beverage composition comprising:
no more than about 0.2% by dry weight catechin; and
no more than about 2% by dry weight amino acid;
wherein said amino acid comprises at least one of 1-proline and 1-arginine;
wherein a source of said catechin comprises at least one of white tea solids and epigallocatechin gallate extract;
wherein said composition comprises a beverage.

8. The functional beverage composition of claim 7 wherein said composition comprises between about 0.1% and about 0.2% by dry weight catechin; and between about 0.9% and about 2% by dry weight amino acid.

9. The functional beverage composition of claim 7 wherein said composition comprises between about 0.8% and about 1.1% by dry weight 1-proline.

10. The functional beverage composition of claim 7 wherein said composition comprises between about 0.09% and about 0.5% by dry weight 1-arginine.

11. The functional beverage composition of claim 7 wherein said composition comprises between about 0.4% and about 0.5% by dry weight white tea solids.

12. The functional beverage composition of claim 7 wherein said composition comprises between about 0.03% and about 0.13% by dry weight epigallocatechin gallate extract.

13. A method for promoting skin health in a consumer, comprising:
orally administering to said consumer an effective amount of a consumable composition comprising:
no more than about 0.2% by dry weight catechin; and
no more than about 2% by dry weight amino acid;
wherein said amino acid comprises at least one of 1-proline and 1-arginine;
wherein a source of said catechin comprises at least one of white tea solids and epigallocatechin gallate extract.

14. The method of claim 13, wherein said consumable composition is administered in combination with one or more additional active ingredients selected from lipoic acid, selenium, vitamin E, vitamin B and vitamin C.

15. The method of claim 13 wherein said consumable composition comprises a beverage.

16. The method of claim 15 wherein said consumable composition comprises between about 0.1% and about 0.2% by dry weight catechin; and between about 0.9% and about 2% by dry weight amino acid.

17. The method of claim 15 wherein said consumable composition comprises between about 0.8% and about 1.1% by dry weight 1-proline.

18. The method of claim 15 wherein said consumable composition comprises between about 0.09% and about 0.5% by dry weight 1-arginine.

19. The method of claim 15 wherein said consumable composition comprises between about 0.4% and about 0.5% by dry weight white tea solids.

20. The method of claim 15 wherein said consumable composition comprises between about 0.03% and about 0.13% by dry weight epigallocatechin gallate extract.

21. A functional consumable composition comprising:
no more than about 0.2% by dry weight catechin; and
at least one of lipoic acid, selenium, vitamin E, vitamin B, and vitamin C;
wherein a source of said catechin comprises at least one of white tea solids and
epigallocatechin gallate extract.

22. The functional consumable composition of claim 21 further comprising no more than about 0.06% by dry weight vitamin E.

23. The functional consumable composition of claim 21 further comprising no more than about 1% by dry weight vitamin C.

24. The functional consumable composition of claim 21 further comprising no more than about 0.0004% by dry weight selenium.

25. A functional consumable composition comprising:
no more than about 0.2% by dry weight catechin; and
no more than about 2% by dry weight amino acid.

26. The functional consumable composition of claim 25 wherein said amino acid comprises at least one of 1-proline and 1-arginine.
